(19) United States
(12) Patent Application Publication
(10) Pub. No.: US 2026/0016445 A1
(43) Pub. Date: Jan. 15, 2026

(54) SALIVARY SIALIC ACID TESTING FOR CANCER DETECTION

(71) Applicant: Newton Howard, Potomac, MD (US)

(72) Inventor: Newton Howard, Potomac, MD (US)

(21) Appl. No.: 19/335,575

(22) Filed: Sep. 22, 2025

Related U.S. Application Data
(63) Continuation-in-part of application No. 18/745,523, filed on Jun. 17, 2024.
(60) Provisional application No. 63/521,645, filed on Jun. 16, 2023.
(60) Provisional application No. 63/698,879, filed on Sep. 25, 2024.

(51) Int. Cl. G01N 27/416; G01N 21/31; G01N 21/65; G16H 10/60; G16H 50/20

(52) U.S. Cl. CPC G01N 27/4166; G01N 21/31; G01N 21/658; G16H 10/60; G16H 50/20

(57) ABSTRACT

The present systems and methods relate to devices, methods, and systems for detecting the probability of cancer conditions using Sialic Acid concentrations. The present invention relates to a non-invasive system with diagnostic and treatment capacities that use a unified code that is intrinsic to physiological brain function. Embodiments may provide salivary sialic acid testing for cancer detection, for example, breast and oral cancers. Findings indicate the effectiveness of salivary sialic acid testing for detecting solid cancers, potentially serving as an initial screening tool in laboratory settings before invasive diagnostic procedures are recommended.

### CROSS-REFERENCE TO RELATED APPLICATIONS

This application claims the benefit of U.S. Provisional Application No. 63/698,879, filed Sep. 25, 2024, and is a continuation-in-part of U.S. patent application Ser. No. 18/745,523, filed Jun. 17, 2024, which claims the benefit of U.S. Provisional Application No. 63/521,645, filed Jun. 16, 2023, the contents of all of which are incorporated by reference herein in their entirety.

### BACKGROUND

The present systems and methods relate to devices, methods, and systems for detecting the probability of cancer conditions using Sialic Acid concentrations.

Cancer is a leading public health problem associated with significant morbidity and mortality worldwide. A recent study found that approximately ten million people died from cancer across the globe in 2020, while the number of new cases was around twenty million. Breast cancer was the most prevalent tumor reported, with the disease increasing in recent years. According to the Global Burden of Disease Report, breast cancer accounted for nearly two million cases in 2017. The disability-adjusted life years attributable to breast cancer were estimated to be 17,708,600.

Cancer screening is critical for early diagnosis and access to therapy. A review estimated that the relative reduction in deaths from breast cancer due to early screening is about 20%. The main method of screening for breast cancer is mammography. However, many women experience barriers to breast cancer screening. For example, women who live in remote rural regions are disproportionately impacted by inequitable access to screening. One study found that they were less likely to get a mammogram compared to their non-rural counterparts.

Although early detection via mammography saves lives, regular use of mammographs has its disadvantages, especially exposure to radiation. False positives from mammograms and the need for invasive tissue biopsies are other disadvantages. While the benefits often outweigh the risks, it might not be feasible for some women to perform mammograms regularly. Younger women (i.e., below the age of forty) are also often excluded from screening as for them the risks outweigh the benefits at the population level. However, this practice may lead to missed cases at an individual level. Although rarer, younger women with breast cancer tend to have more aggressive forms of the disease with a higher mortality. Finding less invasive ways to screen them may thus be advantageous. Furthermore, some women experience delays in getting a mammogram, particularly in regions with lower availability.

Patients with other solid tumors, such as oral cancer, may also experience barriers to screening. Dentists are the first point of contact for screening for oral cancer, and not all patients see them for regular checks, including those designated as “high-risk” in one study. Barriers include traveling for remote patients and the costs associated with dental care.

Finding noninvasive ways to pre-screen or adjunct methods of screening that are simple, affordable, and readily accessible is thus important to reduce the barriers to cancer screening. In recent years, the application of biomarkers, such as salivary sialic acid, has increasingly been evaluated for solid tumors. The collection of saliva provides advantages because it is relatively simple and noninvasive and may be used widely in labs. Home collection kits can be sent to patients living in remote areas. There is thus a need to evaluate whether this method is accurate in detecting breast cancer and other solid tumors. Therefore, the rationale for this perspective arises from the imperative to identify noninvasive and easily accessible modalities for cancer screening, specifically those that can serve as pre-screening or supplementary tools alongside existing methods.

### SUMMARY

The present systems and methods relate to devices, methods, and systems for detecting the probability of cancer conditions using Sialic Acid concentrations. The present invention relates to a non-invasive system with diagnostic and treatment capacities that use a unified code that is intrinsic to physiological brain function. Embodiments may provide salivary sialic acid testing for cancer detection, for example, breast and oral cancers. Findings indicate the effectiveness of salivary sialic acid testing for detecting solid cancers, potentially serving as an initial screening tool in laboratory settings before invasive diagnostic procedures are recommended.

For example, in an embodiment, a computer-implemented method for determining a probability of presence of a cancer condition may comprise: determining a concentration of sialic acid in a saliva sample of a person using a sialic acid concentration analysis device comprising: receiving the saliva sample of the person and processing the saliva sample of the person for analysis, analyzing the saliva sample of the person to determine the sialic acid concentration, and generating digital data representing the concentration of sialic acid in the saliva sample of the person; receiving the digital data representing the concentration of sialic acid in the saliva sample of the person; comparing the determined concentration of sialic acid with a database relating sialic acid concentrations with cancer conditions; and determining and outputting a probability of presence of a particular cancer condition based on the comparison of the determined concentration of sialic acid with a database relating sialic acid concentrations with cancer conditions.

In embodiments, the sialic acid concentration analysis device may comprise a spectrophotometer. The sialic acid concentration analysis device may comprise a Raman spectrometer. The sialic acid concentration analysis device may comprise an electrochemical sensor.

In an embodiment, a system for determining a probability of presence of a cancer condition may comprise: a sialic acid concentration analysis device adapted to determine a concentration of sialic acid in a saliva sample of a person comprising: apparatus configured to receive the saliva sample of the person and process the saliva sample of the person for analysis, apparatus configured to analyze the saliva sample of the person to determine the sialic acid concentration, and circuitry configured to generate digital data representing the concentration of sialic acid in the saliva sample of the person; a computer system comprising a processor, memory accessible by the processor, and program instruction and data stored in the memory whereby the computer system is adapted to: control the sialic acid concentration analysis device so as to obtain the concentration of sialic acid; receive the digital data representing the concentration of sialic acid in the saliva sample of the person, compare the determined concentration of sialic acid with a database relating sialic acid concentrations with cancer conditions, and determine and output a probability of presence of a particular cancer condition based on the comparison of the determined concentration of sialic acid with a database relating sialic acid concentrations with cancer conditions.

### BRIEF DESCRIPTION OF THE DRAWINGS

The details of the present invention, both as to its structure and operation, can best be understood by referring to the accompanying drawings, in which like reference numbers and designations refer to like elements.

FIG. 1 is an exemplary illustration of the difference in concentration of sialic acid in cancer patients and healthy control groups.

FIG. 2a is an exemplary illustration of frequency of sialic acid concentrations.

FIG. 2b is an exemplary illustration of frequency of sialic acid concentrations.

FIG. 3 is an exemplary illustration of association between salivary sialic acid levels and oral cancer.

FIG. 4 is an exemplary illustration of a serum levels of TSA and LSA in oral cancer patients.

FIG. 5 is an exemplary illustration of sialic acid concentration (SAC) of groups of patients.

FIG. 6 is an exemplary flow diagram of cancer detection using Sialic Acid concentration.

FIG. 7 is an exemplary block diagram of a computing device, in which processes involved in the embodiments described herein may be implemented.

FIG. 8 is an exemplary diagram of a process of using a portable Raman spectrometer for cancer detection

FIG. 9 is an exemplary illustration of a network of portable Raman devices.

FIG. 10 is an exemplary block diagram of a system in accordance with the present embodiments

### DETAILED DESCRIPTION

The present invention relates to a non-invasive system with diagnostic and treatment capacities that use a unified code that is intrinsic to physiological brain function. Embodiments may provide salivary sialic acid testing for cancer detection, for example, breast and oral cancers. Findings indicate the effectiveness of salivary sialic acid testing for detecting solid cancers, potentially serving as an initial screening tool in laboratory settings before invasive diagnostic procedures are recommended.

Salivary sialic acid testing may provide early cancer detection and improved healthcare accessibility. Additionally, portable testing technologies may enhance its role in pre-screening individuals, monitoring treatment response, and facilitating timely referrals for more targeted diagnostic procedures. By addressing barriers to cancer screening, such as invasiveness and limited access to healthcare facilities, salivary sialic acid testing has the potential to revolutionize cancer detection and significantly contribute to saving lives and improving patient outcomes.

There is a strong positive relationship between cancer (oral and breast) and salivary sialic acid levels. This data includes a dose-response relationship, with higher levels of the acid being linked to more advanced stages of cancer.

Recent studies have highlighted the potential of salivary sialic acid as a non-invasive biomarker for cancer detection. Elevated levels of sialic acid in saliva have been consistently associated with various types of cancer, including breast, oral, and ovarian cancers. For instance, patients with these cancers exhibit significantly higher salivary sialic acid levels compared to healthy individuals, making it a promising marker for early detection and monitoring disease progression. In breast cancer patients, salivary sialic acid levels were found to be nearly three times higher (18.5 mg/dL) than in healthy controls (6.7 mg/dL). Similarly, oral cancer patients showed elevated levels of sialic acid, with pre-cancerous conditions having levels around 57.5 mg/dL and advanced stages reaching up to 80.4 mg/dL. Ovarian cancer studies also revealed significant differences, with benign cases showing 5.1 mg/dL and malignant cases reaching 23.0 mg/dL.

Salivary sialic acid testing can be a useful method not only to detect cancer and catch early asymptomatic or precancerous stages of the disease but also to monitor the response to treatment. Salivary sialic acid levels declined in patients who had been treated for cancer, so salivary sialic acid may be a good prognostic marker as well as a potential tool to monitor treatment response and disease progression. Furthermore, salivary sialic acid levels may also be useful as pre-screeners for cancer in patients who feel healthy. Following this use, patients may be referred for more diagnostic measures, such as mammograms and tissue biopsies.

By increasing accessibility to timely pre-screening, salivary sialic acid testing could potentially save many lives. Furthermore, mammograms use radiation, which is harmful and can slightly increase the risk of developing cancer in the long run. Thus, women who would otherwise not have undertaken a mammogram (because they belong to a lower-risk group), may find it easier to simply test their saliva for sialic acid levels and then make informed decisions about follow-up imaging and biopsy procedures.

Individuals at risk of oral cancer, including those who chew tobacco or have HPV (which slightly increases the risk of oral tumors), may benefit from simple and noninvasive salivary testing. Dentists are usually the first point of contact for diagnosing oral cancer, but not all patients have equitable access to dental care. By undertaking a simple and noninvasive test, individuals may then gain priority access to oral cancer care, including those who would have avoided visiting the dentist. It is also important to further establish whether both free and bound salivary sialic acid levels should be evaluated, given the potential for false positives in tobacco chewers without cancer. Bound levels may be significantly different in those with cancer versus those without, suggesting that this may be a more accurate measure.

Further, salivary sialic acid testing may be used for detecting ovarian cancer and other solid tumors. After hypothetically using this form of testing, patients can make informed decisions based on their family history, risk factors, and symptoms on whether to undertake further confirmatory and specification tests.

Another important aspect is the threshold for normal levels versus the mean level that indicates an abnormality. Typically, higher levels are found in patients with cancer, especially at later stages. However, it is important to determine diagnostic criteria and variation across the population according to race, ethnicity, and sex. Given the promising results discussed above, it would be beneficial to make this form of testing more available to patients at their medical clinics.

For the assay of free, protein-bound, and/or total salivary SA, techniques such as variations of spectrophotometric assays employing either Gaitonde's acid ninhydrin reagent reaction developed by Yao and colleagues or Skoza and Mohos protocol, which uses periodate, sodium arsenite, and thiobarbituric acid. These methods were developed in 1970-1980s and are well-known for the analysis of SA, and now can be considered reference techniques for detection of this biomarker in saliva.

While quite precise and chemically simple, these methods do have certain disadvantages. Number and complexity of manipulations, special chemical reagents, as well as the need for spectrophotometer to read the final result, make these protocols useable only by trained professionals in a clinical lab setting.

Other detection techniques, such as surface enhanced Raman spectroscopy technique may be used reliably analyze SA in patients. For example, use may be made of surface-enhanced Raman scattering signal of SA absorbed on nanoparticles to generate analytic signal using Raman spectrometer. The method allows much more facile analysis of SA in saliva. Instead of a sequence of chemical manipulations, saliva sample aliquot is just mixed with the nanoparticle solution in specific proportions. This method has great potential to replace quite dated Yao and Skoza+Mohos protocols, offering a low cost and simple assay technique, using a Raman spectrometer.

Example of other techniques that may be used include an electrochemical sensor for highly sensitive SA detection was based on molecularly imprinted polymers, it displayed good selectivity, reproducibility and stability. Such method for SA detection can be portable and adapted for personalized use, as no sample processing is required. A sensor for SA based on optic fibers modified by magnetic nanoparticles was also developed, response time of which amounts to a few seconds, which is orders of magnitude faster than gold standard techniques, which can take up to 40 min per sample. Another state-of-the art device, a capillary sensor based on UiO-66-NH2 metal-organic framework, which has shown operation in micro-volume (15 μl) analysis of SA in a rapid, reliable fashion. All these and many other recent developments in novel SA sensing techniques are yet to find application in clinical validation studies. However, these exciting developments show great promise for translation of salivary SA assay to more portable solutions, which would not require trained professional attention, and ultimately may be used by patients themselves in home settings for potentially real-time analysis of SA levels in saliva for early cancer detection.

Commonalities among the Lowry method, Navazesh method, Ninhydrin method, Diphenylene method, and Yao method include their use of spectrophotometry to measure color changes in the samples. The Lowry method and Navazesh method both measure the amount of protein in the sample by inducing a color change reaction and measuring the resulting color using a spectrophotometer. The Ninhydrin method, Diphenylene method, and Yao method are specifically focused on measuring the amount of sialic acid (SA) in the sample. They all involve the addition of specific chemicals to the sample to induce a color change reaction, which is then measured using a spectrophotometer. In terms of potential for portable or wearable devices, the Yao method may be more suitable. It utilizes Gaitonde's acid ninhydrin reagent and spectrophotometry to measure the color change in the sample. This method has the potential for implementation in portable spectrophotometers, allowing for on-the-go analysis of sialic acid levels. However, it is important to note that the suitability of these methods for portable or wearable devices depends on various factors such as the size, complexity, and power requirements of the spectrophotometric equipment used. Further research and development are needed to optimize these methods for portable or wearable applications, including the exploration of miniaturized spectrophotometry or alternative sensing techniques like electrochemical or optical sensors.

Raman spectrometers have made significant progress towards becoming more portable and handheld, while spectrophotometers require more advanced laboratory infrastructure. Currently, Raman spectrometers have more potential for on-site or in-field analysis. However, both technologies are advancing, and future developments could lead to more portable versions of both instruments.

Both Raman spectrometry and spectrophotometry can provide accurate results for saliva analysis, depending on the specific application and method used. Raman spectrometry offers higher accuracy and specificity for specific molecular analyses, while spectrophotometry is more suitable for overall protein or DNA content measurements.

The surface enhanced Raman spectroscopy technique, as well as developments in molecularly imprinted polymers and optic fibers modified by magnetic nanoparticles, show promise for portable and rapid salivary sialic acid detection. These advancements have potential for future clinical validation studies.

A portable device that can accurately measure salivary sialic acid levels offers a compelling solution to the limitations of current screening methods. Such a device would provide a non-invasive, cost-effective, and easily accessible means of cancer detection, enabling timely diagnosis and intervention. By integrating advanced Raman spectrometry with microfluidic technology, this portable device aims to deliver high sensitivity and specificity in detecting sialic acid levels, thereby facilitating early cancer detection and improving healthcare outcomes, particularly in low-resource settings.

A portable Raman spectrometry device for detecting salivary sialic acid levels may be designed to meet the rigorous demands of accurate, rapid, and non-invasive cancer screening. To achieve this, the device may be structured into two distinct modules, each responsible for different stages of the measurement process. This modular design enhances the device's functionality, efficiency, and user experience.

Exemplary specifications for a portable Raman spectrometry device may include: a Minimum Detection Limit: ≤0.01 mg/dL; Resolution: 0.01 mg/dL (ideal 0.001 mg/dL); Accuracy: ±0.1 mg/dL; Portability: Weight <1 kg, dimensions ≤15 cm×15 cm×10 cm; Sample Volume: 1-5 mL; Measurement Time: ≤10 minutes; Power Supply: Battery-operated, ≥8 hours battery life; Data Storage and Connectivity: Storage for 1000 measurements, wireless connectivity (Bluetooth or Wi-Fi); User Interface: Intuitive interface with clear display and minimal user input; Calibration and Quality Control: Automated calibration routines and quality control checks.

To ensure the portable Raman spectrometry device meets the necessary requirements for effective cancer detection via salivary sialic acid measurement, several technical tasks and specifications may be described:

Minimum Detection Limit: Requirement: The device must be capable of detecting sialic acid concentrations as low as 0.01 mg/dL. Purpose: Ensures sensitivity to low levels of sialic acid, which is critical for early detection of cancer.

Resolution: Requirement: The device should have a resolution of at least 0.01 mg/dL, with an ideal resolution of 0.001 mg/dL. Purpose: High resolution is necessary to distinguish between slight variations in sialic acid concentrations, improving diagnostic accuracy.

Accuracy: Requirement: The device should achieve an accuracy within ±0.1 mg/dL. Purpose: Ensures reliable and consistent measurements, which is essential for clinical decision-making.

Portability: Requirement: The device should be lightweight (less than 1 kg) and compact (dimensions not exceeding 15 cm×15 cm×10 cm). Purpose: Enhances usability in various settings, including remote and resource-limited environments.

Sample Volume: Requirement: The device should process saliva samples of 1-5 mL. Purpose: Ensures sufficient sample volume for accurate measurements without requiring excessive saliva collection. Measurement Time: Requirement: The device should complete the measurement process within 10 minutes. Purpose: Provides rapid results, improving patient throughput and convenience.

Power Supply: Requirement: The device should be battery-operated with a battery life of at least 8 hours of continuous use. Purpose: Ensures operation in locations without a reliable power source and enhances portability.

Data Storage and Connectivity: Requirement: The device should have onboard data storage for at least 1000 measurements and wireless connectivity (Bluetooth or Wi-Fi) for data transfer. Purpose: Facilitates data management and integration with healthcare systems.

User Interface: Requirement: The device should feature an intuitive interface with a clear display and minimal user input required. Purpose: Simplifies operation, reducing the likelihood of user error and ensuring accessibility for non-technical users.

Calibration and Quality Control: Requirement: The device should include automated calibration routines and quality control checks. Purpose: Maintains measurement accuracy and reliability over time.

The decision to separate the device into two modules stems from the need to optimize both the mechanical and electronic processes involved in the detection and analysis of salivary sialic acid. By compartmentalizing these processes, the sensitive electronic components are ensured to be isolated from potential mechanical vibrations and sample contamination, thereby enhancing the device's overall performance and reliability.

### Module A: Sampling, Filtration, and Centrifugation

### Responsibilities:

Sample Collection: This module begins the process with the collection of saliva samples using a specialized collection kit that ensures sample integrity and stability.
Filtration: It then filters the collected sample to remove any debris and large particles, ensuring that only the necessary components reach the measurement stage.
Centrifugation: The filtered sample undergoes centrifugation to separate and concentrate the salivary components, making it ready for precise analysis.

### Benefits:

Efficiency: By handling all pre-processing steps in one module, it streamlines the sample preparation process.
Contamination Control: Keeping mechanical processes separate from sensitive electronics reduces the risk of contamination and damage.

### Module B: Sensing and Electronics

### Responsibilities:

Measurement: This module receives the pre-processed sample and performs the Raman spectrometry analysis. It houses the laser, spectrometer, and CCD detector required for high-sensitivity and high-resolution measurements.
Data Processing: It processes the Raman spectra to identify and quantify the sialic acid levels, using advanced data processing and analysis techniques.
User Interface: This module also includes the user interface, data storage, and connectivity features, allowing users to interact with the device and manage the measurement data effectively.

### Benefits:

Precision: By isolating the sensitive measurement and processing components, this module ensures high accuracy and reliability of the results.
Usability: Integrating the user interface and connectivity in this module enhances the overall user experience, making the device more intuitive and efficient to use.

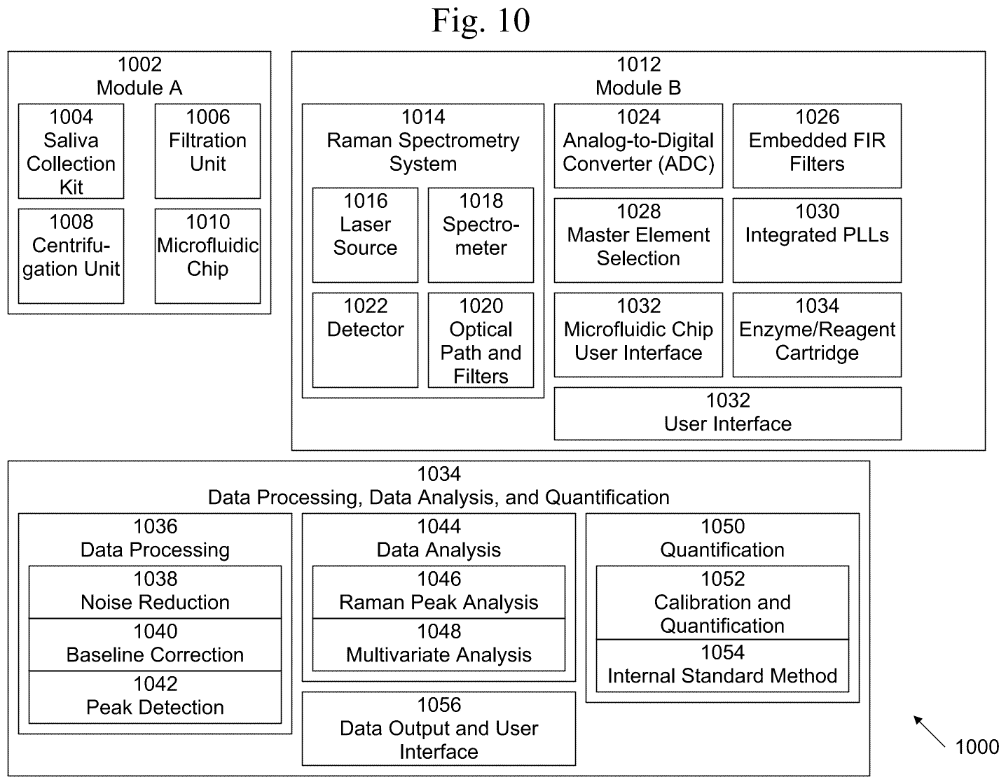

FIG. 10 is an exemplary block diagram of a system 1000 in accordance with the present embodiments.

Module A: Sampling, Filtration, and Centrifugation 1002. Module A 1002 is the initial stage of the device, responsible for preparing the saliva sample for analysis. This module includes the saliva collection kit, filtration unit, centrifugation unit, and microfluidic chip. Each component plays a crucial role in ensuring the sample is adequately prepared and delivered to the measurement module for accurate and reliable analysis.

Saliva Collection Kit. 1004 Purpose: The saliva collection kit 1004 is designed to collect and preserve the saliva sample from the patient. This kit ensures that the sample remains uncontaminated and stable until it undergoes further processing. Parameters and Elements: Components: Collection tube and stabilization solution. Reasoning: The collection tube provides a sterile environment for collecting the sample, preventing contamination. The stabilization solution preserves the integrity of the saliva, preventing degradation of sialic acid and other components. Volume Capacity: 1-5 mL. Reasoning: This volume range is sufficient to ensure enough sample is available for accurate measurement without requiring excessive collection effort from the patient.

Filtration Unit. 1006 Purpose: The filtration unit 1006 removes debris and large particles from the saliva sample, ensuring that only the relevant biological components are processed further. This step is critical for preventing blockages and ensuring accurate analysis. Parameters and Elements:

Pore Size: 0.22 μm (PTFE filter). Reasoning: A pore size of 0.22 μm is small enough to remove most debris and microorganisms while allowing the passage of sialic acid and other analytes. PTFE (Polytetrafluoroethylene) is chosen for its chemical resistance and non-reactive nature, ensuring no interference with the sample. Flow Rate: 1-5 mL/min. Reasoning: This flow rate is optimized to provide efficient filtration without causing excessive pressure drop, ensuring smooth sample processing.

Centrifugation Unit. 1008 Purpose: The centrifugation unit 1008 separates and concentrates the components of the saliva sample, making it ready for precise analysis. This step enhances the detection sensitivity by isolating the sialic acid from other components. Parameters and Elements: RCF (Relative Centrifugal Force): 2000×g. Reasoning: An RCF of 2000×g is sufficient to effectively separate the saliva components, concentrating the sialic acid for better detection sensitivity. RPM (Revolutions Per Minute): 6000-8000 RPM (depending on rotor radius). Reasoning: The RPM is chosen based on the rotor radius to achieve the desired RCF. This speed range is effective for saliva samples, ensuring efficient separation without damaging the sample. Sample Volume Capacity: 5 mL. Reasoning: This capacity matches the collection kit volume, ensuring the entire sample can be processed without requiring multiple steps.

Microfluidic Chip. 1010 Purpose: The microfluidic chip 1010 handles the precise movement and mixing of the sample and reagents, ensuring accurate delivery to the measurement chamber. It provides a controlled environment for sample processing, minimizing contamination and sample loss. Parameters and Elements: Material: PDMS (Polydimethylsiloxane) with hydrophilic coating. Reasoning: PDMS is biocompatible and optically transparent, making it ideal for biological applications. A hydrophilic coating improves fluid flow and prevents non-specific adsorption of biomolecules. Channel Dimensions: 100 μm width×100 μm height. Reasoning: These dimensions ensure efficient handling of small saliva volumes, maintaining laminar flow, which is crucial for precise control of fluid movement. Flow Rate: 1-5 L/min. Reasoning: This flow rate allows slow and controlled movement of the sample through the channels, preventing sample loss and ensuring accurate delivery to the measurement chamber. Pumps/Valves: Piezoelectric micropump and microvalves. Reasoning: Piezoelectric micropumps and microvalves provide precise control over the sample movement, ensuring consistent flow rates and accurate sample delivery.

Module B 1012: Sensing and Electronics. Module B 1012 is responsible for the precise measurement and analysis of the pre-processed saliva sample. This module includes the Raman spectrometry system, ADC, microcontroller, microfluidic chip, enzyme/reagent cartridge, and the data processing, analysis, and quantification techniques. Each component is critical for ensuring the accuracy, reliability, and usability of the device.

Raman Spectrometry System 1014. Purpose: The Raman spectrometry system 1014 is designed to detect and measure the concentration of sialic acid in the saliva sample using Raman scattering principles. It provides high sensitivity and specificity for accurate analysis.

Components and Parameters: Laser Source 1016, Wavelength: 785 nm. Reasoning: This wavelength reduces fluorescence interference in biological samples, providing clearer Raman signals. Power Output: 50 mW. Reasoning: Sufficient to excite Raman scattering without damaging the sample or causing excessive heating. Beam Quality (M²): Close to 1. Reasoning: Ensures a high-quality, focused beam essential for efficient Raman scattering. Stability: Power fluctuation less than 1%. Reasoning: Ensures consistent excitation and reliable measurements, minimizing variability in the Raman signal.

Spectrometer 1018. Spectral Resolution: 1 cm⁻¹. Reasoning: High spectral resolution allows for precise identification of Raman peaks, ensuring accurate detection and quantification. Detection Range: 100-4000 cm⁻¹. Reasoning: Covers the Raman spectra of most biomolecules, including sialic acid, providing comprehensive analysis. Grating: High-efficiency grating (1200 lines/mm). Reasoning: Maximizes signal collection efficiency, improving the signal-to-noise ratio (SNR). Detector: Type: Charge-Coupled Device (CCD). Reasoning: CCDs are highly sensitive and have low noise, making them ideal for detecting the weak signals characteristic of Raman scattering. Cooling: Thermoelectric cooling (TEC). Reasoning: Cooling the CCD reduces thermal noise, enhancing sensitivity, especially in low-light applications like Raman spectrometry. Pixel Size: 15 μm. Reasoning: Smaller pixels provide higher spatial resolution, important for resolving fine spectral features.

Optical Path and Filters 1020: Notch or Edge Filters: High-quality optical filters 1020 to block Rayleigh scattered light (e.g., Semrock RazorEdge 785 nm). Reasoning: Essential for isolating the weak Raman signal from the intense excitation light. Beam Splitter: High transmission efficiency at the laser wavelength and high reflectance at the Raman shifted wavelengths (e.g., Thorlabs BSF10-A). Reasoning: Ensures that the majority of the excitation light reaches the sample while efficiently directing the Raman scattered light to the detector 1022. Objective Lens: High numerical aperture (NA) lens, typically NA>0.7 (e.g., Olympus MPlanFL N 50×/0.75 NA). Reasoning: A high NA lens collects more scattered light, improving the efficiency and sensitivity of the system. Optical Alignment: Precision alignment of optical components. Reasoning: Ensures maximum signal collection efficiency and minimizes signal loss, leading to more accurate and reliable measurements.

Analog-to-Digital Converter (ADC) 1024: Model: AD7760 (250 ksps with extremely low INL and embedded FIR filters). Purpose: The ADC 1024 converts the analog signals from the Raman spectrometer into digital data for processing. Parameters and Elements: Resolution: 24-bit. Reasoning: High resolution ensures precise digitization of the Raman signal, capturing subtle differences in intensity. Sampling Rate: 250 ksps. Reasoning: High sampling rate captures the dynamic range of the Raman signal, ensuring accurate data reconstruction. INL (Integral Non-Linearity): Extremely low. Reasoning: Low INL is critical for accurate representation of the signal over the entire input range, ensuring precise quantitative analysis.

Embedded FIR Filters 1026: Reasoning: Improves signal-to-noise ratio by filtering out unwanted frequencies, enhancing the quality of the digitized signal.

Master Element Selection 1028. Model: Spartan-7 FPGA. Purpose: The Spartan-7 FPGA manages the operation of the sensors, processes the measurement data, and performs necessary calculations. It replaces a traditional microcontroller due to its superior computational capabilities and flexibility, which are essential for the complex and high-speed data processing required in Raman spectrometry. Parameters and Elements: Clock Frequency: Up to 400 MHz. Reasoning: The high clock frequency allows the FPGA to perform rapid computations and handle high-speed data from the ADC. This is crucial for real-time processing of Raman spectrometry data, ensuring timely and accurate measurements.

Phase-Locked Loops (PLLs) 1030: Integrated PLLs 1030. Reasoning: PLLs 1030 provide precise clock management and synchronization, ensuring stable and efficient operation of the FPGA and its subsystems. This stability is essential for maintaining accurate timing and data integrity during high-speed processing. Logic Cells: Over 200,000 logic cells. Reasoning: A large number of logic cells provides the necessary computational power to handle complex data processing algorithms. This includes noise reduction, baseline correction, peak detection, and multivariate analysis, all of which require significant processing resources. DSP Slices: Up to 120 DSP slices. Reasoning: DSP slices are critical for performing high-speed mathematical operations, such as those required for signal processing tasks. These include filtering, Fourier transforms, and other computationally intensive tasks that are integral to Raman spectrometry data analysis. Block RAM: Up to 1,024 Kbits of block RAM. Reasoning: Adequate block RAM ensures efficient storage and quick access to intermediate data during processing. This is essential for handling large data sets and performing real-time computations without bottlenecks.

Microfluidic Chip 1032: Purpose: The microfluidic chip handles the precise movement and mixing of the sample and reagents, ensuring accurate delivery to the measurement chamber. Parameters and Elements: Material: PDMS (Polydimethylsiloxane) with hydrophilic coating. Reasoning: PDMS is biocompatible and optically transparent, making it ideal for biological applications. A hydrophilic coating improves fluid flow and prevents non-specific adsorption of biomolecules. Channel Dimensions: 100 μm width×100 μm height. Reasoning: These dimensions ensure efficient handling of small saliva volumes, maintaining laminar flow, which is crucial for precise control of fluid movement. Flow Rate: 1-5 μL/min. Reasoning: This flow rate allows slow and controlled movement of the sample through the channels, preventing sample loss and ensuring accurate delivery to the measurement chamber. Pumps/Valves: Piezoelectric micropump and microvalves. Reasoning: Piezoelectric micropumps and microvalves provide precise control over the sample movement, ensuring consistent flow rates and accurate sample delivery.

Enzyme/Reagent Cartridge 1034. Purpose: The enzyme/reagent cartridge 1034 contains the necessary reagents for the Raman measurement of sialic acid. It may include enzymes or other chemicals that react with sialic acid to produce a measurable signal. Parameters and Elements: Reagent Type: Sialidase enzyme. Reasoning: Sialidase specifically cleaves sialic acid, producing a product that can be detected more easily by Raman spectroscopy. Reagent Stability: Stable at room temperature for up to 6 months. Reasoning: Ensures the reagents remain active and effective over time, especially important for portable devices. Reagent Delivery System: Integrated microchannels within the microfluidic chip. Reasoning: Ensures accurate and consistent mixing of reagents with the sample, which is crucial for reliable measurements. Volume Capacity: 20 μL per reaction. Reasoning: Minimizes reagent waste and ensures efficient reactions within the microfluidic system. Type: Disposable cartridge. Reasoning: Prevents cross-contamination and simplifies the user experience by eliminating the need for cleaning and maintenance.

User Interface 1036. Purpose: The user interface 1036 allows the operator to interact with the device, initiate measurements, and view the results. Components and Parameters: LED Display. Type: Small LCD or OLED screen. Size: Compact display suitable for a portable device. Purpose: To display the sialic acid concentration results and provide user instructions. Commands: Start Measurement: Initiates the measurement process. Function: The user presses a button to start the sample processing and Raman measurement. View Results: Displays the final concentration of sialic acid. Function: The user can press a button to view the sialic acid levels in mg/dL, along with additional information such as sample ID and measurement date/time. Calibration: Initiates a calibration routine. Function: The user can trigger a calibration process to ensure the device maintains accuracy. Diagnostic Check: Runs a system diagnostic. Function: The user can initiate a self-check to verify that all components are functioning correctly. Settings: Accesses device settings. Function: The user can configure various device parameters, such as display brightness and power settings. Final Result Display: Sialic Acid Concentration: Displayed in mg/dL. Example: “Sialic Acid: 12.5 mg/dL”. Sample ID: Identifies the specific sample being measured. Example: “Sample ID: 00123”. Measurement Date/Time: Indicates when the measurement was taken. Example: “Date/Time: 2024-07-20 14:35”. Diagnostic Messages: Provides information on device status or errors. Example: “Status: Calibration Required”.

Data Processing, Data Analysis, and Quantification 1034: Data Processing 1036. Noise Reduction 1038: Technique: Savitzky-Golay Filter. Reasoning: This filter 1038 smooths the data by fitting successive subsets of adjacent data points with a low-degree polynomial, preserving peak shapes while reducing random noise. Parameters: Polynomial order (e.g., 2 or 3), window size (e.g., 11 or 21 data points). FPGA Role: The FPGA applies the Savitzky-Golay filter to the incoming digital data from the ADC. It processes each subset of data points, performs the polynomial fitting, and outputs the smoothed data. Data Flow: Analog signals→ADC→Digital data→FPGA→Smoothed digital data. Baseline Correction 1040: Technique: Asymmetric Least Squares (ALS) Smoothing. Reasoning: Corrects the baseline by fitting a smooth baseline to the data, effectively removing background fluorescence and other baseline drifts. Parameters: Smoothness parameter (λ), asymmetry parameter (p). FPGA Role: The FPGA applies ALS smoothing to the smoothed digital data. It iteratively adjusts the baseline, using the specified parameters to fit a smooth curve that removes background interference. Data Flow: Smoothed digital data→FPGA→Baseline-corrected data.

Peak Detection: Technique 1042: Continuous Wavelet Transform (CWT). Reasoning: Enhances features of interest and is particularly effective for identifying small and overlapping peaks in Raman spectra. Parameters: Wavelet function (e.g., Mexican hat or Haar), scale range for detection. FPGA Role: The FPGA performs CWT on the baseline-corrected data to detect peaks. It identifies the positions and scales of the peaks, enhancing their features for further analysis. Data Flow: Baseline-corrected data→FPGA→Peak-enhanced data.

Data Analysis 1044: Raman Peak Analysis 1046: Technique: Curve Fitting (Gaussian or Lorentzian Fitting). Reasoning: Essential for accurately determining the position, height, and width of Raman peaks. Gaussian and Lorentzian fits model the shape of Raman peaks. Parameters: Initial guesses for peak positions, widths, and heights. FPGA Role: The FPGA fits Gaussian or Lorentzian models to the detected peaks. It calculates the optimal parameters for each peak, providing detailed information about their shape and intensity. Data Flow: Peak-enhanced data→FPGA→Fitted peak data. Multivariate Analysis 1048: Technique: Principal Component Analysis (PCA). Reasoning: Reduces data dimensionality while preserving variance, helping to identify significant features in the Raman spectra and distinguish between sample types. Parameters: Number of principal components to retain. FPGA Role: The FPGA performs PCA on the fitted peak data. It extracts the principal components, reducing the data to its most significant features for classification and analysis. Data Flow: Fitted peak data→FPGA→Principal components.

Quantification 1050: Calibration and Quantification 1052: Technique: Partial Least Squares Regression (PLSR). Reasoning: Suitable for developing calibration models that correlate Raman spectral data with sialic acid concentration. It handles multicollinearity well and is effective in quantitative analysis. Parameters: Number of latent variables, cross-validation method. FPGA Role: The FPGA uses PLSR to correlate the principal components with sialic acid concentrations, applying pre-calibrated models to determine the concentration in the sample. Data Flow: Principal components→FPGA→Sialic acid concentration. Internal Standard Method 1054: Technique: Use of Internal Standards. Reasoning: Adding an internal standard to the sample improves accuracy by compensating for variations in sample volume and instrument conditions. The ratio of the sialic acid peak intensity to the internal standard peak intensity is used for quantification. Parameters: Choice of internal standard, concentration of the internal standard. FPGA Role: The FPGA calculates the ratio of sialic acid peak intensity to the internal standard peak intensity, adjusting the quantification accordingly. Data Flow: Sialic acid concentration→FPGA→Adjusted concentration based on internal standard

Data Output and User Interface 1056: Step 1: The FPGA formats the final data, including the sialic acid concentration and any additional diagnostic information. Step 2: The formatted data is sent to the display interface. Step 3: The results are presented on a small LCD or OLED screen in a user-friendly format, showing the sialic acid concentration in mg/dL, along with sample ID, measurement date/time, and any diagnostic messages.

### Sialic Acid Concentration and Its Correlation with Various Cancers, Cancer Stages, and Classifications

The realm of cancer encompasses a range of diseases characterized by abnormal cell growth, which can infiltrate organs and metastasize to other parts of the body. Cancerous cells have the potential to damage surrounding healthy tissues, resulting in diverse symptoms depending on the specific type of cancer. While some cancers exhibit more readily identifiable symptoms, others present greater challenges for early detection. The mortality rate associated with cancer is alarmingly high, with 18.1 million new cases reported in 2018 and 9.6 million resulting in death. However, certain cancers prove more fatal than others due to various factors, including the effectiveness of disease detection and treatment. For instance, brain cancer demonstrates a meager 5-year survival rate of 12.2%, oral cancer stands at 50%, while testicular cancer boasts an impressive 5-year survival rate of 95.3%.

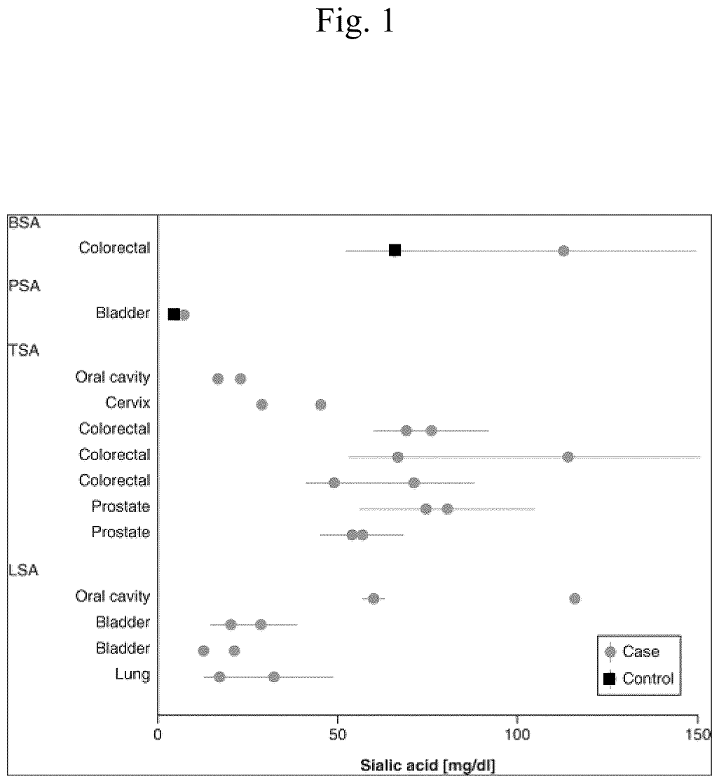

Research encompassing diverse malignancies has suggested that sialic acid may serve as a promising indicator and marker for the presence of these malignancies. Numerous studies across different disciplines have highlighted increased levels of sialic acid in patients compared to healthy individuals, thereby identifying total sialic acid (TSA), bound sialic acid (BSA), and protein-bound sialic acid (PSA) as potential markers for various types of cancer. FIG. 1 is a 2-D plot displaying the difference in concentration of sialic acid in cancer patients and healthy control groups. Moreover, alterations in sialylation levels have been observed in siglecs and other receptors such as EGFR60-62 in lung and colorectal cancers.

TABLE 1
Comparison between sialic acid levels in cancer patients and control groups.

| No. of | Cancer | | | | |
|---|---|---|---|---|---|
| participants | type | Control(mg/dl) | Disease(mg/dl) | p-value | Ref |
| 107 | Lung | LSA: 17.2 (12.7; 21.7) | LSA: 32.4 (16.0; 48.8) | N/A | 47 |
| 118 | Bladder | LSA: 20.3 (14.5; 26.1) | LSA: 28.7 (22.4; 38.9) | 0.001 | 48 |
| | | PSA: 5.6 (4.4;6.7) | PSA: 7.2 (5.9; 8.5) | | |
| 67 | Breast | TSA: 83.6 | TSA: 108 | <0.01 | 49 |
| 60 | Oral | TSA: 29.0 (28.2; 29.8) | TSA: 45.3 (43.8; 46.8) | 0.05 | 50 |
| | | LSA: 16.7 (16.1; 17.4) | LSA: 23.0 (22.1; 23.9) | | |
| 91 | Cervix | TSA: 60.0 (56.9; 63.1) | TSA: 108 | <0.01 | 51 |
| 540 | Prostate | TSA: 54.1 (45.1; 63.1) | TSA: 56.8 (45.3; 68.3) | 0.013 | 52 |
| 111 | Colorectal | TSA: 66.8 (53.1; 80.5) | TSA: 114.1 (77.3; 150.9) | <0.0001 | 53 |
| | | BSA: 65.9 (52.3; 79.5) | BSA: 112.9 (76.1; 149.7) | | |

LSA-lipid-bound sialic acid,
TSA-total sialic acid,
BSA-bound sialic acid,
PSA-protein-bound sialic acid

### Sialic Acid and Breast Cancer

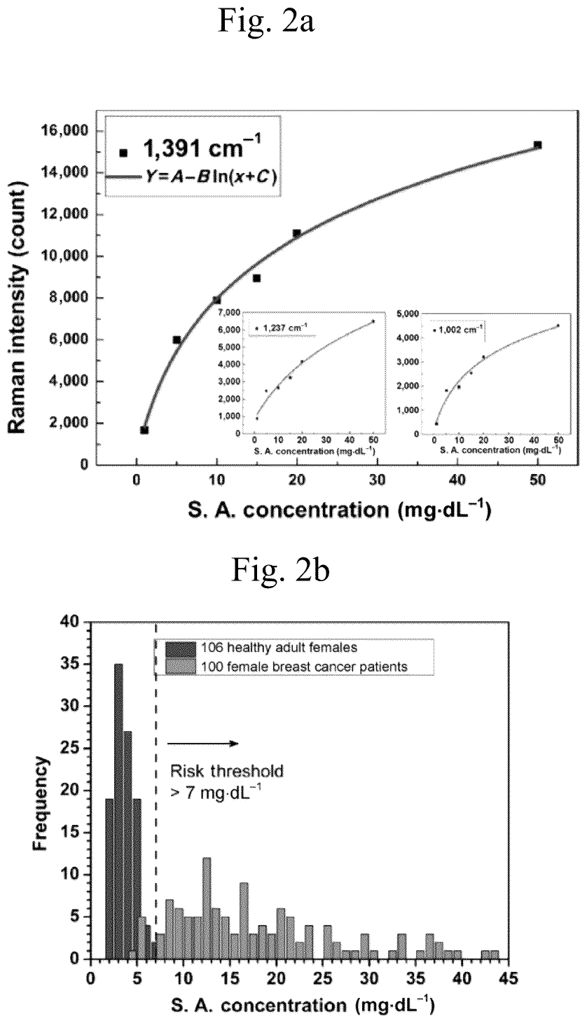

A few studies evaluated the relationship between salivary sialic acid levels and breast cancer. These studies found significantly higher sialic acid levels in patients with breast cancer compared to cancer-free individuals. FIG. 2 shows a plot of frequency of sialic acid concentrations with an interval width of 1 mg·dL⁻¹ for both healthy controls (red bars) and patients with breast cancer, irrespective of cancer stage (green bars).Breast cancer was confirmed in patients using mammography followed by tissue biopsy. The study by Artega et al. found that sialic acid levels were nearly twice as high in patients, with a mean concentration of 14.9 mg/dl versus 6.7 mg/dl (p-value<0.01). This study validated salivary sialic acid, finding a sensitivity of 80% and specificity of 93%. Another study by the authors, which compared levels in 100 breast cancer patients versus 164 healthy individuals, found that the mean salivary sialic acid levels were 18.5 mg/dl in patients versus 3.5 mg/dl in healthy subjects. A study conducted in Turkey by Ozturk et al. found that the mean salivary sialic acid level was 114 mg/dl in patients compared to 47 mg/dl in healthy individuals (p-value<0.01). It should be noted that this study was undertaken in patients with established and pre-diagnosed breast cancer at different stages, which affected the mean level, rather than participants who were screened for cancer during the early stages of the disease.

Cancer biomarker sialic acid: Several studies have evaluated the relationship between salivary sialic acid (SA) levels and breast cancer. These studies found significantly higher SA levels in patients with breast cancer compared to cancer-free individuals. A study by Artega et al. found that SA levels were more than twice as high in cancer patients, with a mean concentration of 14.9 mg/dL versus 6.7 mg/dL (p<0.01). This study validated salivary SA as a biomarker, finding a sensitivity of 80% and specificity of 93%. Another study by the authors, which compared levels in 100 breast cancer patients versus 164 healthy individuals, found that the mean salivary SA levels were 18.5 mg/dL in cancer patients versus 3.5 mg/dL in healthy subjects. A study conducted in Turkey by Ozturk et al. found that the mean salivary SA level was 114 mg/dL in cancer patients compared to 47 mg/dL in healthy individuals (p<0.01). It should be noted that this study was undertaken in patients with established and pre-diagnosed breast cancer at different stages, which affected the mean level, rather than in participants who were screened for cancer only during the early stages of the disease.

### Sialic Acid and Oral Cancer

### Staging and Malignancy Indicator

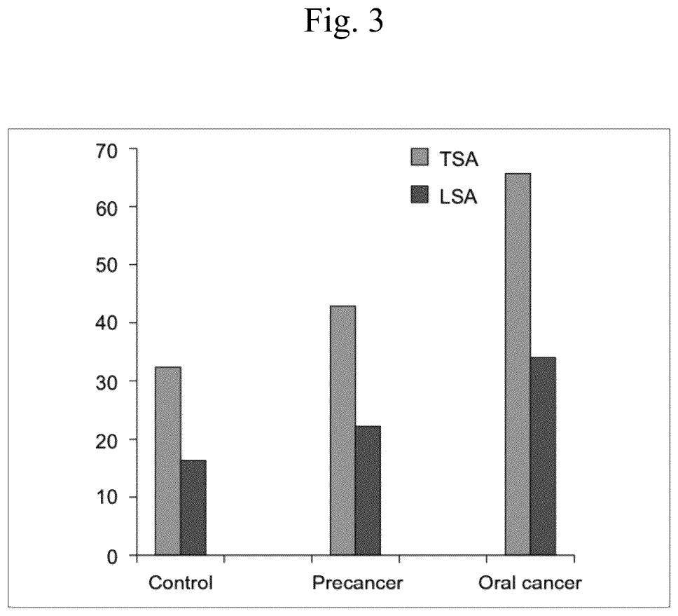

Most of the studies on salivary sialic acid have been undertaken in patients with oral cancer. These studies have all found a significant and positive association between salivary sialic acid levels and oral cancer. FIG. 3 shows the elevation of TSA and LSA levels exhibited statistical significance among untreated oral cancer patients when compared to the control and precancer groups. This suggests that serum glycoconjugates have the potential to serve as a distinct differentiator between oral cancer and precancer conditions.

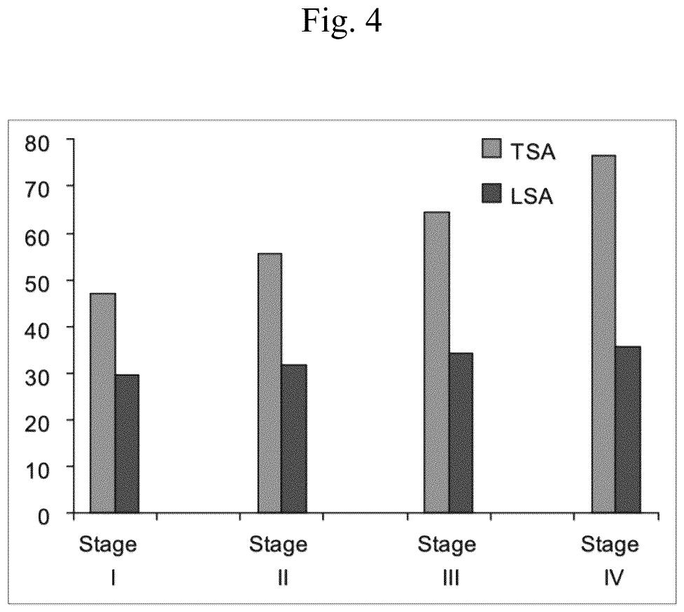

A dose-response relationship was also identified, with higher sialic acid levels linked to more advanced stages of cancer and progression. For example, the study by Hemalatha et al. in India found that salivary sialic acid levels were directly associated with the histopathological grade of the carcinoma, with lower levels in less differentiated tumors compared to more advanced tumor grades. Specifically, patients with well-differentiated oral tumors had mean salivary sialic acid levels of 7.4 mg/dl when compared to patients with less differentiated tumors (6.6 mg/dl) (p-value<0.01); healthy controls had mean levels of 2.1 mg/dl. Similarly, there was a positive relationship between mean salivary sialic acid levels and cancer staging, including marked differences in mean levels in patients with pre-cancerous tumors (57.5 mg/dl) compared to healthy controls (40.3 mg/dl) and patients with established oral cancer (80.4 mg/dl) (p-value<0.01). FIG. 4 shows serum levels of TSA and LSA in oral cancer patients, which demonstrate a progressive increase from stage-I to stage-IV. The highest levels are observed in the most advanced stage of malignancy, indicating a direct correlation with tumor burden, primary lesion stage, and presence of distant metastasis. It should be noted that the study conducted by Poudel et al. in Nepal did not find a significant association between moderately differentiated and late-stage squamous cell carcinomas and salivary sialic acid levels. However, the differences were still significant in patients with squamous cell carcinoma compared to healthy controls.

Further studies identified higher SA levels linked to more advanced stages of cancer and progression. For example, a study by Dadhich et al. in India found that salivary SA levels were significantly enhanced in a group of 30 oral cancer patients when compared to a group of 30 gender and age matched healthy individuals (9 mg/dL versus 1.5 mg/dL). A similar result was obtained in an independent study with 50 oral cancer patients that were compared to 50 healthy control persons. Furthermore, there was a positive relationship between mean salivary SA levels and cancer staging, including marked differences in mean levels in patients with precancerous tumors (57.5 mg/dL) compared to healthy controls (40.3 mg/dL) and patients with established oral cancer (80.4 mg/dL) (p<0.01).

Trivedi et al. investigated the effects of chemotherapy on salivary SA levels in patients with oral cancer and found that patients who had undergone chemotherapy had significantly lower levels compared to cancer patients who had not been treated. Furthermore, Poudel et al. found a significant negative relationship between salivary SA levels and treatment duration.

Finding noninvasive ways to prescreen or adjunct methods of screening that are simple, affordable, and readily accessible is thus important to reduce the barriers to cancer screening. The collection of saliva provides advantages because it is relatively simple and noninvasive and maybe widely used in labs. Home collection kits can be sent to patients living in remote areas. Thus, there is a need to evaluate whether this method is accurate in detecting breast cancer and other solid tumors. Therefore, the rationale for this perspective arises from the imperative to identify noninvasive and easily accessible modalities for cancer screening, specifically those that can serve as prescreening or supplementary tools alongside existing methods.

### Bound Versus Free Salivary Sialic Acid Levels

The study by Hemalatha et al. only found an association with bound salivary sialic acid levels when compared to free levels and oral cancer. However, another study conducted in India, by Sanjay et al., found that both free and bound salivary sialic acid levels were associated with oral cancer, with a dose-response relationship. The study by Azeem et al. found that both tobacco chewers and non-tobacco chewers with oral cancer had increased levels of bound salivary sialic acid, with no significant differences between the two groups. However, oral cancer patients had significantly higher levels of free salivary sialic acid relative to their tobacco-chewing counterparts without cancer (p-value<0.05). Thus, assessing both free and bound levels may be useful in tobacco-using populations (r=−0.5; p-value<0.05). In another study using Kruskal Wallis test significant difference in serum and salivary levels of sialic acid between control, tobacco user, PML, and OSCC groups was demonstrated (P=<0.0001).

Table 2 shows significant differences between the four groups in salivary and serum Free Sialic Acid (FSA) and Protein-based Sialic Acid (PBSA).

| | | | | | | | Mann-Whitney U test |
|---|---|---|---|---|---|---|---|
| | | Min | Max | | Chi | | and Bonferroni's |
| Category | N | (mg/dl) | (mg/dl) | Median | Square | P Value | correction test) |
| SalPBSA | Control | 1.00 | 4.00 | 2.10 | 31.34 | <.0001 | Control Vs Other + |
| | Tobacco | 3.70 | 5.90 | 4.40 | | | Tobacco user Vs Oscc |
| | user | | | | | | P < .0001 |
| | PML | 2.10 | 9.40 | 5.20 | | | |
| | OSCC | 4.01 | 7.90 | 5.80 | | | |
| SalPSA | Control | .09 | 3.78 | 1.80 | 24.40 | <.0001 | PML/OSCC Vs Control + |
| | Tobacco | 1.40 | 4.80 | 2.10 | | | PML/OSCC Vs tobacco |
| | user | | | | | | users P < .01 |
| | PML | 2.23 | 4.80 | 3.70 | | | |
| | OSCC | 2.30 | 5.81 | 3.81 | | | |

### Effects of Chemotherapy

The study by Trivedi et al. investigated the effects of chemotherapy on salivary sialic acid levels in patients with oral cancer, finding that patients who had undergone chemotherapy had significantly lower levels compared to cancer patients who had not been treated. Furthermore, Poudel et al. found a significant negative relationship between salivary sialic acid levels and treatment duration.

### Sialic Acid and Other Solid Tumors

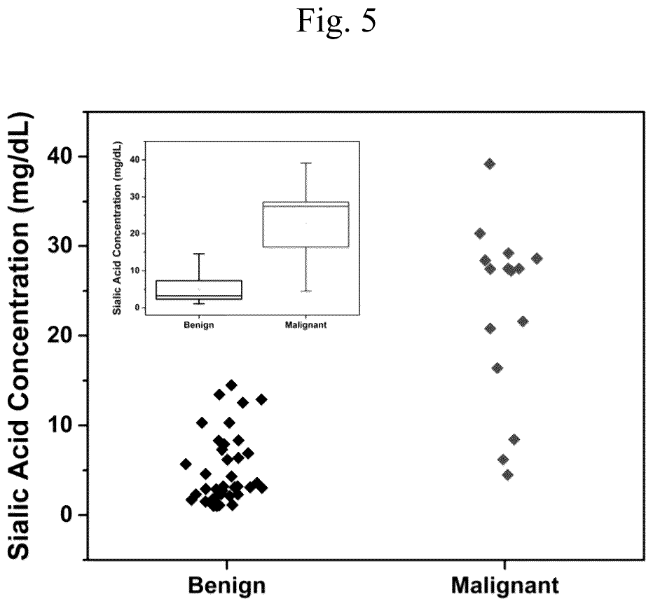

In addition to oral and breast cancers, one study explored the relationship between salivary sialic acid levels and ovarian cancer. The study was conducted by De Jesus et al. in Mexico and found a significant difference in salivary sialic acid levels in benign compared to malignant ovarian cancers. The reported range of sensitivity and specificity was 80-100%, indicating that Sialic Acid levels measured from saliva can be equally good predictor as MRI index for the presence of ovarian cancer in sensitivity while outperforming it in terms of specificity. FIG. 5 shows the sialic acid concentration (SAC) of each group of patients. On the left are the SAC of benign adnexal mass (BAMP) affected patients, and on the right those in which histology has diagnosed ovarian cancer (OCP).

Sialic acid quantification methods: Due to its potential to serve as a biomarker in various diseases, a plethora of methods have been developed to quantify free SAs. Common to all methods is the fact that SA is first released from the cell surface. On one hand, this can be done using various acidic compounds. The released SA is subsequently derivatized with a colorimetric agent or with a fluorescent molecule to be detected and quantified. The disadvantage of these methods is that derivatization is not specific to SAs; as such, the measurements are overrated with respect to SA levels.

In contrast to these traditional methods, recent advancements have introduced more sophisticated techniques like Raman spectroscopy and biosensors.

Raman spectroscopy, particularly in its Surface-Enhanced Raman Scattering (SERS) form, offers a label-free, highly sensitive approach to sialic acid detection. This technique relies on the inelastic scattering of photons, providing a molecular fingerprint of the sample. On the other hand, biosensors typically involve labeled detection strategies, incorporating specific receptors or antibodies for sialic acid. While Raman spectroscopy excels in its minimal sample preparation and rapid analysis capabilities, biosensors are noted for their specificity and potential for quantification in complex biological matrices. The choice between these methodologies depends on factors such as the required sensitivity, the complexity of the sample matrix, and the need for label-free detection.

To overcome this limitation, chromatographic methods, such as high performance liquid chromatography, can be applied to separate labeled SAs from interfering molecules. On the other hand, the release of SAs can be triggered by enzymatic digestion. Again, the SA is then derivatized to be labeled by various methods and quantified. The enzymatic method has the advantage of resulting in measurements with high specificity; however, the process is slow and cumbersome.

More recent developments include SA biosensors with the assistance of multiwalled carbon nanotubes and ferrocene or the biosensor enhanced by near-infrared light excitation and localized surface plasmon resonance. Most promising is the development of Raman-spectroscopic methods, such as the one developed by Hernández-Arteaga as it provides highly sensitive and specific results. However, all these methods require sophisticated and large machines and trained personnel in a laboratory setting and are, as such, not suited for an easy-access screening program. It is therefore of paramount importance that portable devices be engineered that would allow for individual sample collection and processing while maintaining the result interpretation for a trained professional in a medical research laboratory.

Notably, most clinical research on SA levels in saliva and their connection to various types of cancer has been carried out using classical lab based approaches, such as Yao's method. These methods are well developed and produce excellent results. However, they require the presence of medical professionals and lab-trained personnel to assess SA levels. Preferably, screening for cancer should be done as early as possible, and at home solutions would be perfect for that goal. Translation of classical lab based techniques to mobile, point-of-care, and personal solutions does not seem to be feasible, mainly because they involve dangerous chemicals, are bulky, and require expensive equipment for analysis.

State-of-the-art research on sialic acid analysis has produced intriguing solutions, such as wearable sensors for SA and miniaturized electrochemical sensors. While these exciting developments show excellent performance in SA detection, they are rarely employed in real samples of actual cancer patients.

On the other hand, some developments go hand-in-hand with human studies. For example, a novel SERS-based method to detect SA levels in saliva was developed, which was validated with patients with cancer or other diseases. It was used to uncover new links between SA and various diseases, such as breast cancer. Raman spectroscopy is already widely used in various handheld mobile applications, and one can easily imagine that the Raman method for SA quantification can also become a mobile device.

There is strong evidence to support the use of salivary SA to prescreen individuals for cancer, including oral and breast cancers. While the ‘Sialic Acid Quantification Methods’ section detailed the technical aspects of Raman spectroscopy and biosensors, we now turn our attention to their broader implications in cancer detection. Raman spectroscopy, particularly SERS, offers a promising, rapid, and non-invasive approach for cancer diagnostics, potentially revolutionizing point-of-care testing. Its label-free nature reduces preparation time and preserves the integrity of the sample. However, challenges exist in its application in complex biological systems and ensuring specificity. Biosensors, known for their high specificity, could complement Raman spectroscopy by providing targeted detection, especially in heterogeneous samples. Integrating these technologies could pave the way for more comprehensive diagnostic platforms. Future research should focus on overcoming the limitations of each method and exploring their combined potential in clinical applications.

The reviewed studies reinforce a strong positive relationship between cancer stage (oral and breast) and salivary SA levels. Salivary SA testing is not only useful for detecting cancer and identifying early asymptomatic or precancerous stages but also for monitoring treatment responses. As levels decline in treated patients, salivary SA could serve as a prognostic marker and a tool for monitoring treatment effectiveness and disease progression. Additionally, salivary SA levels might be valuable as prescreeners for cancer in asymptomatic patients, leading to more diagnostic measures like mammograms and tissue biopsies.

By increasing accessibility to timely prescreening, salivary SA testing could save many lives. Furthermore, mammograms use radiation, which is harmful and can slightly increase the risk of developing cancer in the long run,. Thus, women who would otherwise not have undertaken a mammogram (because they belong to a lower-risk group) may find it easier to simply test their saliva for SA levels and then make informed decisions about follow-up imaging and biopsy procedures.

Individuals at risk of oral cancer, including those who chew tobacco or have human papillomavirus (which slightly increases the risk of oral tumors), may benefit from simple and noninvasive salivary testing. Dentists are usually the first point of contact for diagnosing oral cancer, but not all patients have equitable access to dental care. By undertaking a simple, noninvasive test, individuals may gain priority access to oral cancer care, including those who would have avoided visiting the dentist. It is also important to further establish whether both free and bound salivary SA levels should be evaluated, given the potential for false positives in tobacco chewers without cancer found in this review. One study found that bound levels were significantly different in those with cancer versus those without, suggesting that this may be the preferred measure.

More research is needed to better understand whether salivary SA testing may be used to detect ovarian cancer, as one study supported its potential applicability. Further evaluation of salivary SA in relation to other solid tumors is also of interest. After hypothetically using this form of testing, patients can make informed decisions based on their family history, risk factors, and symptoms regarding whether to undertake further confirmatory and specification tests.

Different ranges of salivary SA levels and supported evidence that higher levels are found in patients with cancer, especially at later stages. However, it is important to determine reliable cut-off levels for the diagnostic criteria and to establish whether the levels vary across populations according to race, ethnicity, and sex. Given the promising results discussed above, it would be beneficial to make this form of testing more available to patients at medical clinics.

For the assay of free, protein-bound, and/or total salivary SA, most scoped studies use variations of spectrophotometric assays employing either Gaitonde's acid ninhydrin reagent reaction developed by Yao and colleagues or Skoza and Mohos' protocol, which uses periodate, sodium arsenite, and thiobarbituric acid. These methods were developed in the 1970s-1980s and are well-knownfor SA analysis, and some are still considered to be reference techniques for detection of this biomarker in saliva.

While quite precise and chemically simple, these methods have certain disadvantages. The number and complexity of manipulations, special chemical reagents, and the need for a spectrophotometer to read the final results make these protocols useable only by trained professionals in a clinical lab setting.

New and portable SA quantification methods: All the above-mentioned SA quantification methods are qualified to be designed into a portable device. Chemical reactions can be performed in micro vessels, and the addition of substances can be controlled and driven within microfluidic channels and microvalves. Photometers and fluorescence readers for detection have already been miniaturized and can be combined with the necessary chemical reaction chambers. Nevertheless, portable devices that measure SA should encompass a quantification method that is reasonably fast, specific, and sensitive. Such methods include surface-enhanced Raman spectroscopy, for which various approaches have been studied. One advantage of this methodology lies in its minimized sample preparation steps. Furthermore, various types of biosensors, would also lend themselves to portable devices.

While salivary SA analysis has great potential for the pre-diagnostic detection of cancers, methodological complexity may preclude the introduction of such screening in everyday clinical analysis, and even more so in patient-side and/or personalized use. For example, a molecularly imprinted polymers based sensor was reported to have high selectivity, stability, sensitivity and reproducibility. Such a method for SA detection can be portable and adapted for personalized use, as no sample processing is required.

A sensor for SA based on optic fibers modified by magnetic nanoparticles was also developed, with a response time of a few seconds, which is significantly faster than gold standard techniques, which can take up to 40 min per sample. Another state-of-the art device, a capillary sensor based on UiO-66-NH2 metal-organic framework, has shown operation in microvolume (15 μl) analysis of SA in a rapid, reliable fashion. These and many other recent developments in novel SA sensing techniques have yet to find applications in clinical validation studies. However, these exciting developments show great promise for the translation of salivary SA assays to more portable solutions, which would not require trained professional attention, and ultimately may be used by patients themselves in home settings for potentially real-time analysis of SA levels in saliva for early cancer detection.

Raman spectroscopy is a technique that measures the scattering of laser light by molecules within a sample. It provides valuable information about the chemical composition and molecular structure of the analyzed substance. It can identify specific chemical bonds and functional groups and even detect trace amounts of substances. Raman spectroscopy is widely used for qualitative and quantitative analyses in various fields, including pharmaceuticals, forensics, materials science, and environmental monitoring.

In terms of portability, both portable spectrophotometers and portable Raman spectrometers are designed for on-site or field applications. However, portable Raman spectrometers are typically more compact and handheld,, allowing for easy mobility and measurements in real time at the point of analysis.

A portable Raman spectrometer is particularly suitable for SA analysis, providing molecular-specific information for the identification and analysis of specific compounds, including SA. Significant advancements have been made in the precise detection of SA in untreated saliva using Raman spectroscopy, specifically SERS. Moreover, studies involving cancer patients have successfully utilized this technology. Given the ease of use and widespread adoption of handheld Raman spectrometers, they have promising potential for portable SA detection in the future.

Nevertheless, there are a few things to consider when aiming at using Raman spectroscopy to determine SA levels to predict cancer disease status. Raman spectroscopy is mainly used as a qualitative analytical method whereby the Raman shifts are specific to a given analyte; an example of

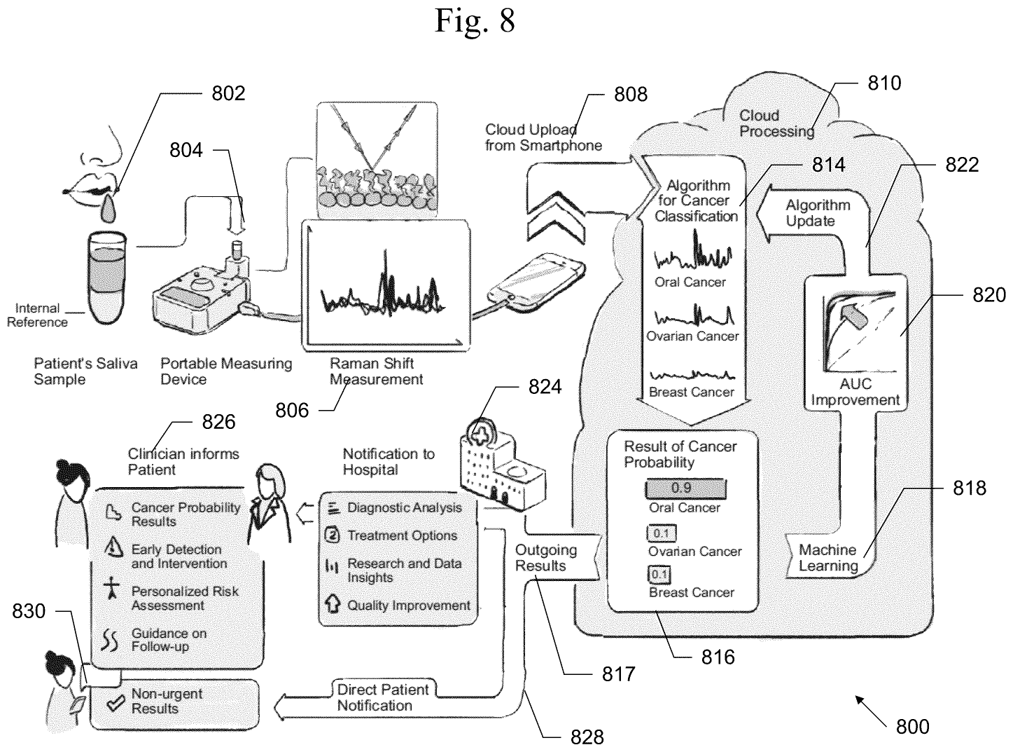

Raman shifts obtained via a portable SERS device is schematically shown in FIG. 8. Quantitative statements based on the area under the curve are more difficult to make, as these statements are dependent on the amount of incoming electromagnetic waves and the medium in which the analyte resides. The composition of saliva is different from person to person, and thus poses a problem in interpreting the results quantitatively5. To correct for these differences, a specific, yet to be determined, internal reference of known amount must be added to the saliva samples5. Also, the analytical volume must be normalized to obtain reproducible results. We envisage a device in which the patient's saliva is collected. By closing the device, pressure would be applied to the saliva sample such that it passes through a rough filter that would retain particulate matter into a well with a defined volume. The well would contain the internal standard substance in dried form (like a tablet) and would be solubilized when in contact with the saliva sample (FIG. 8).

FIG. 8 outlines an exemplary process of using a portable Raman spectrometer for cancer detection. It starts with the collection of saliva samples, and then shows how the portable Raman spectrometer is employed, highlighting the application of a laser to SA, which leads to the generation of spectrum plots containing Raman shift curves. Lastly, the diagram demonstrates the integration of cloud computing, enabling the provision of personalized health advice tailored to medication intake or the necessity of consulting a clinician. The figure highlights the entire process, commencing with saliva collection through a portable Raman spectrometer and smartphone, ending with a personalized feedback for the user. Notably, the proposed portable device will be web-connected to transmit Raman shifts for more accurate analysis, updating the machine learning algorithm online. Subsequently, a probability of potential cancer disease will be conveyed, along with a comprehensive report, to the hospital/clinic. A clinician will then directly contact the patient to inform them of the results and discuss next steps. In cases where the results are favorable and no urgent outcome is indicated, a message will be directly sent to the user.

Process 800 it begins with 802, in which a patient's saliva sample is collected. At 804, the collected saliva sample is input to a portable measuring device, which may include a Raman spectrometer. At 806, the Raman spectrometer measures a Raman shift of the sample and output the measurement results to a communications device, such as a smartphone, for upload to the Cloud 808. At 810, processing of the measurement results may be performed in the Cloud. Processing 810 may include processing the measurement results with one or more algorithms, such as Artificial Intelligence algorithms, to generate classification probability results relating to the presence and type of cancer 814. At 816, the classification probability results may be output 817, and may be used to train one or more machine learning models 818, which may be used to provide improvement to the Area Under the Curve (AUC) 820. Such improvement may provide improvement to the overall accuracy of the diagnostic test. Those improvements may be used to update the detection algorithm 822, so as to improve the detection accuracy. The output classification probability results 817 may be communicated to hospitals or other medical providers 824 for dissemination to the patient 826. Likewise, output classification probability results 817 may be communicated directly to the patient 828, for example, in the case of non-urgent results 830.

FIG. 8 is an illustration of a portable Raman spectrometer for cancer detection.

Raman spectrometry has the potential to yield precise results in saliva analysis, although the selection between the two methods relies on the particular application and technique employed. Raman spectrometry stands out for its exceptional accuracy and specificity in conducting molecular analyses, making it an ideal choice for in-depth molecular characterization.

Challenge 1: instrumentation size and complexity of internal reference The current state of traditional SERS systems poses several challenges that hinder their widespread adoption and usability. These challenges include their bulky and complex nature, as they often require sophisticated laser sources and spectrometers, making them less suitable for portable and point-of-care applications.

Another challenge in performing SERS measurements arises from the need for the selection and quantity of one or more reference substances. This consideration is essential for the standardization of quantitative analysis.

Additionally, sample preparation methods play a critical role in SERS applications, especially in point-of-care settings. Simplifying and standardizing sample preparation methods, such as using a Y-shaped cup or, as proposed in FIG. 1, placement at the bottom of the test tube, is essential for enhancing the practicality and reproducibility of SERS measurements.

To address these challenges and enable more user-friendly and accessible SERS technology, efforts are being made to connect biosensors to smartphones. This offers the potential to create portable and user-friendly platforms for real-time analysis and diagnostics with the ability to capture and analyze Raman spectra conveniently on mobile devices.

Challenge 2: sensitivity and specificity In the realm of cancer detection, achieving high sensitivity and specificity in a portable SERS device is of paramount importance. Accurate and reliable cancer detection relies on the system's ability to discern subtle changes in SA levels associated with cancer while avoiding false positives, which poses a significant challenge.

To address this challenge and enhance the capabilities of portable SERS devices for cancer detection, advanced machine learning algorithms and data analysis techniques have come into play. These powerful tools can be applied to develop robust and accurate cancer classification models based on Raman spectra; a visualization of this process is shown in FIG. 1.

One approach to explore is the utilization of deep learning methods, which can extract complex patterns and relationships from Raman spectral data, potentially leading to improved detection accuracy. Another avenue worth investigating is feature selection, in which specific relevant features are identified and utilized to enhance the discriminative power of classification models. By integrating cutting-edge machine learning and data analysis techniques into portable SERS devices, we can unlock their full potential as powerful tools in the fight against cancer.

Challenge 3: costs Foremost among these challenges is the cost factor, with the current price of handheld SERS systems exceeding $20,000 (https://optosky.com/portableraman. html). Developing a portable version of the SERS system without compromising its sensitivity and accuracy requires substantial investment in research and development. Engineers and scientists need to find innovative solutions to streamline the manufacturing process and utilize cost-effective materials and analysis software without compromising the system's performance. By successfully reducing the cost of the SERS system, its adoption in clinical settings can be significantly enhanced, bringing the promise of early cancer detection closer to reality for a broader population of patients.

Challenge 4: integration and adoption While the scientific merits of SERS based testing are evident, the successful integration into existing healthcare infrastructure demands a multifaceted approach, as shown in FIG. 1. The introduction of a new diagnostic to take the place of existing methods has been historically difficult, however COVID-19 has expanded telehealth and diagnostics (both point of care and at home testing) creating opportunities for other tests to be introduced. Clinical evidence is often not enough for widespread adoption into clinical practice, but also requires stakeholder input, education and training, familiarity and cost effectiveness. Ensuring that stakeholders and users understand the long-term benefits of SA testing will be paramount to its adoption. Adoption of a new cancer diagnostic will require industry engagement and partnership with federal programs such as cancer screening and prevention paradigms to promote implementation. Endorsements from organizations such as national cancer groups and advocacy within the medical community in tandem with commercial availability and cost effectiveness have been shown to have population-level impact.

Opportunity 1: personalized medicine The proposed SERS-based cancer detection portable device has immense potential for revolutionizing the landscape of personalized cancer treatment. It is very likely that additional cancer biomarker(s) will be identified in saliva and possibly in other easily accessible body fluids such as urine in the near future. The combinations of all biomarker measurements can make cancer diagnostics much more discriminative and as such will support personalized patient treatment. Alternatively, with the use of advanced machine learning capabilities, Raman shift patterns of saliva may prove unique for a cancer type, grade and stage and can also indicate patient specific properties important to the therapeutic treatment similar to its use as surgery guidance tool to distinguish cancer cell from healthy cells.

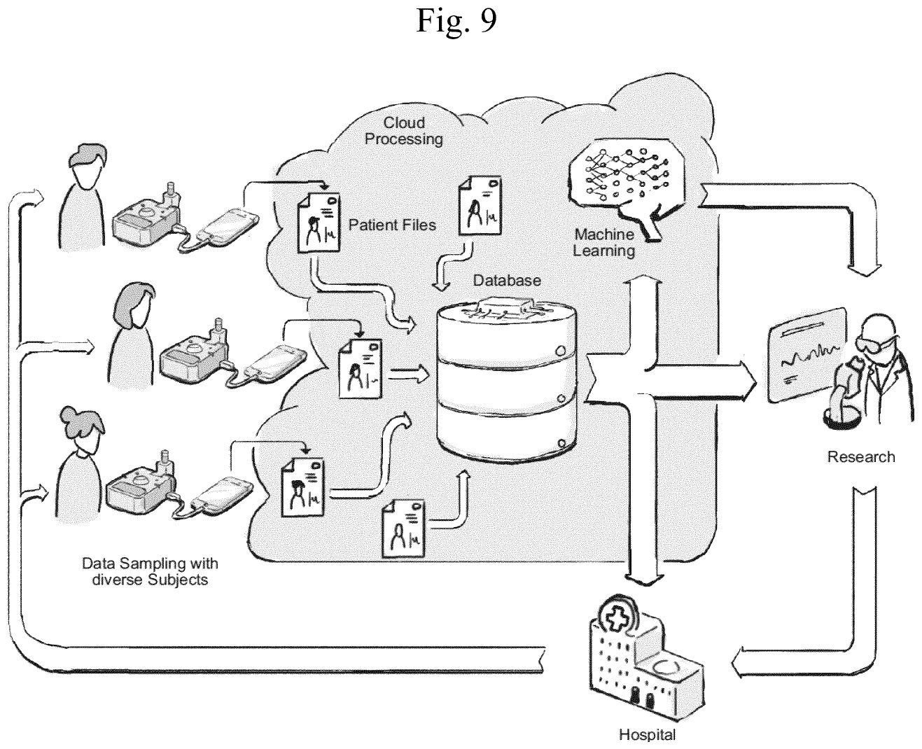

By leveraging such point-of-care SERS-based cancer detection devices, medical professionals can make more informed and precise treatment decisions tailored to individual patient's needs. Personalized treatment strategies have been shown to improve treatment outcomes, minimize adverse effects, and enhance the overall quality of life for cancer patients. These benefits have been observed across various ethnicities and age matched populations, highlighting the universal applicability of personalized approaches (shown in FIG. 9).

Opportunity 2: multiplexing capabilities The possibility of portable SERS technology with multiplexing capabilities presents a groundbreaking advancement in cancer diagnosis, with the potential to transform the field of oncology. By enabling the simultaneous detection of multiple biomarkers in saliva, such as those associated with breast cancer, oral cancer, and ovarian cancer, the diagnostic power of the device is significantly enhanced. A visualization of this process is shown in FIG. 8.

Portable SERS devices equipped with multiplexing capabilities offer a minimally invasive and rapid means of obtaining a comprehensive molecular profile of a patient's tumor, aiding in the early detection and precise characterization of the cancer. This multifaceted approach not only streamlines the diagnostic process, but also provides crucial insights into the tumor's molecular composition, enabling oncologists to tailor personalized treatment plans based on the specific cancer subtype and individual patient characteristics. Moreover, the portability of the SERS system empowers healthcare professionals to conduct on-site diagnostics, even in resource limited or remote settings, thereby improving access to accurate and timely cancer screening.

Opportunity 3: remote collaboration via a network Portable SERS devices offer the potential to bridge geographical gaps and facilitate remote collaboration between healthcare providers and specialists, revolutionizing the way expert opinions and second consultations are sought. With these devices in hand, healthcare professionals can capture detailed molecular information from a patient's tumor sample and transmit the data securely to specialists in other locations. This enables remote experts to analyze SERS spectra and provide valuable insights and recommendations for diagnosis and treatment options. Such collaborative efforts can improve diagnostic accuracy and ensure that patients receive the most appropriate and tailored care, regardless of their geographic location.

To fully realize the potential of portable Raman spectroscopy in cancer detection, FIG. 9 illustrates how extensive clinical studies and validation trials are essential. Collaboration with clinical researchers and oncologists can facilitate the evaluation of the device's performance in large and diverse patient cohorts, encompassing different cancer types and stages. This process, shown in FIG. 9, will provide valuable data on the diagnostic accuracy, sensitivity, and specificity of portable SERS devices, ensuring their reliability and effectiveness in real-world clinical scenarios. Additionally, the inclusion of diverse populations and different ethnicities in these studies will enhance the generalizability of the technology, enabling equitable and effective cancer detection across various patient demographics. Through rigorous clinical translation and validation, portable SERS devices can solidify their role as transformative tools in cancer diagnostics and contribute to improving patient outcomes on a global scale.

Opportunity 4: longitudinal monitoring The ability to monitor simultaneous detection of SA and potential other biomarkers over an extended period would be a game-changer in understanding disease progression, treatment responses, and potential recurrence. For example, AI can be applied to healthcare in predicting patient hospital readmissions to identifying patterns in complex medical datasets offering a glimpse into AI's potential in biomarkers testing. However, for this to be a viable plan, patients receiving treatment must understand the importance of regular testing while also going through a process they find convenient and non-intrusive. Further areas of opportunity with salivary biomarkers monitoring could also be integrated into digital health platforms.

After discussing the potential of AI in predicting patient hospital readmissions and identifying patterns in complex medical datasets, it's important to further clarify the specific role of AI in relation to SA and its potential in biomarker discovery. While SA is a known biomarker for oral cancer, and its detection might often require only setting a diagnostic threshold based on the intensity of SA-specific peaks, AI can play a more nuanced role in this context. Particularly, AI and machine learning algorithms can be crucial in correlating spectral data with patient outcomes, which involves a more complex analysis than simply measuring SA levels.

This capability of AI extends beyond mere quantification of SA. By analyzing vast datasets, AI can help in identifying new patterns and correlations, potentially leading to the discovery of additional biomarkers related to oral and other cancers. In this respect, the use of SERS combined with AI analytics should not be viewed solely as a tool for SA quantification or as a ‘black-box’ for cancer screening. Instead, its value lies in the potential to expand the scope of biomarker identification, offering a more comprehensive understanding of cancer pathogenesis and progression.

Therefore, the integration of AI in longitudinal monitoring, particularly when combined with SERS, opens new avenues for the identification and validation of additional biomarkers. This expanded approach could further refine cancer screening and diagnostic methods, moving beyond the current focus on SA alone. Future research and development in this area could greatly enhance the utility of salivary biomarkers in cancer detection, offering more personalized and precise diagnostic tools.

The digital health platforms that are currently available that track fitness, nutrition, or chronic diseases often employ gamification, rewards, or community support to engage users. These strategies could be adapted for salivary biomarkers testing, specifically with a SERS system. While salivary biomarkers testing can significantly shift the diagnostic and monitoring paradigm, it is just one piece in the larger puzzle of comprehensive cancer care. Patient education, counseling, post-diagnosis care, and continued research into treatment modalities are equally vital,. Personality engagement is also relevant in longitudinal monitoring. For example, beyond generic reminders, personalized messages based on a patient's data might offer more compelling motivation. For instance, visualizing how their salivary biomarkers change over time, correlated with lifestyle choices, could be insightful. The case is similar with community building as establishing patient communities where individuals can share experiences, ask questions, and offer support can bolster long-term engagement.

Salivary biomarkers, particularly sialic acid testing, hold significant promise in the field of cancer detection. Our research emphasizes the potential of this method, especially when combined with advancements in portable Raman spectroscopy and AI analysis. These technologies can enhance the accuracy and convenience of salivary SA testing, potentially revolutionizing early cancer detection. However, the full potential of salivary biomarkers can only be realized through rigorous research and consistent patient engagement. Our focus remains on refining these methods to ensure they are not only scientifically sound but also patient-friendly, facilitating early diagnosis and effective management of cancer.

Outlook The SERS technique, as well as developments in molecularly imprinted polymers and optic fibers modified by magnetic nanoparticles, show promise for portable and rapid salivary SA detection. These advancements have the potential for future clinical validation studies. Recommendations for enhancing cancer detection using portable salivary SA testing are as follows: 1. Continue investigating the relationship between Raman shifts and cancer for oral, ovarian, and breast cancers. Explore the potential of salivary SA testing for detecting additional cancer types. 2. Determine specific and reliable Raman shift features using machine learning for SA to establish diagnostic criteria for different cancer grades and stages and different populations based on race, ethnicity, and sex. 3. Validate and compare different measurement techniques such as SERS technique, molecularly imprinted polymers, and optic fibers modified by magnetic nanoparticles for portable salivary SA analysis. 4. Increase awareness and accessibility of portable salivary SA testing. 5. Carry out large-scale clinical validation to determine the effectiveness of portable testing. 6. Develop portable SERS devices with the ability to detect multiple biomarkers in saliva (in addition to sialic acid) simultaneously, such as those associated with different cancer types. Multiplexing can enhance diagnostic power and streamline the screening process. 7. Enable remote collaboration between healthcare providers and specialists by integrating portable SERS devices into a network.

Salivary sialic acid analysis holds great promise as a non-invasive and early diagnostic tool for various cancers, including oral and breast cancers. Classical lab-based approaches have yielded excellent results in research, but their translation to portable, point-of-care solutions poses challenges due to complexity and costly equipment. State-of-the-art research on SA analysis, including wearable sensors and miniaturized electrochemical sensors, requires further validation with real cancer samples. Portable Raman spectroscopy, particularly surface-enhanced Raman spectroscopy, shows remarkable potential for SA detection. Overcoming challenges related to device size, complexity, cost, while ensuring sensitivity and specificity is essential. Portable SA testing offers opportunities for personalized medicine, multiplexing, and remote collaboration among healthcare providers. Largescale clinical validation is vital for establishing its reliability. Embracing and advancing portable SA testing can significantly enhance cancer screening, early detection, and personalized treatment options.

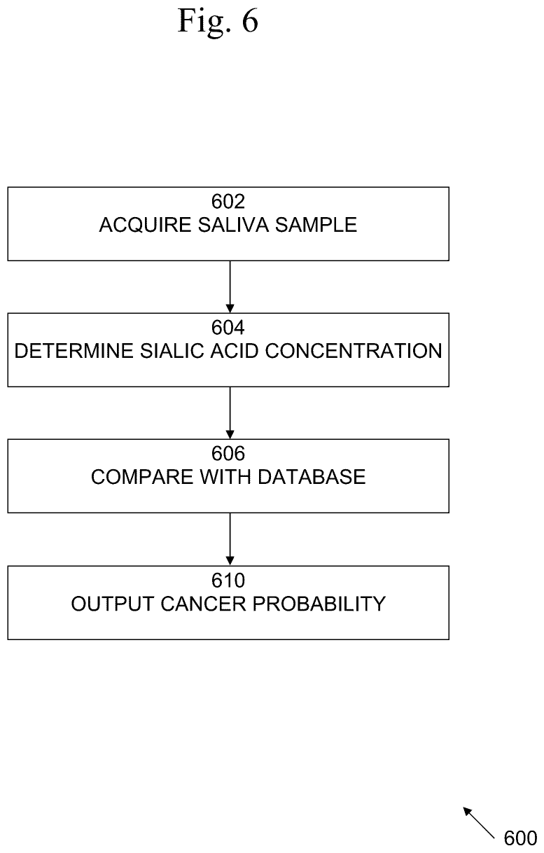

An exemplary process 600 of cancer detection using Sialic Acid concentration is shown in FIG. 6. Process 600 begins with 602, in which a saliva sample may be acquired. At 604, the sialic acid concentration may be determined using one of the techniques described above, or other suitable technique. At 606, the determined sialic acid concentration may be compared with a database relating sialic acid concentrations with cancer conditions. At 610, the probability of presence of particular cancer conditions may be output.

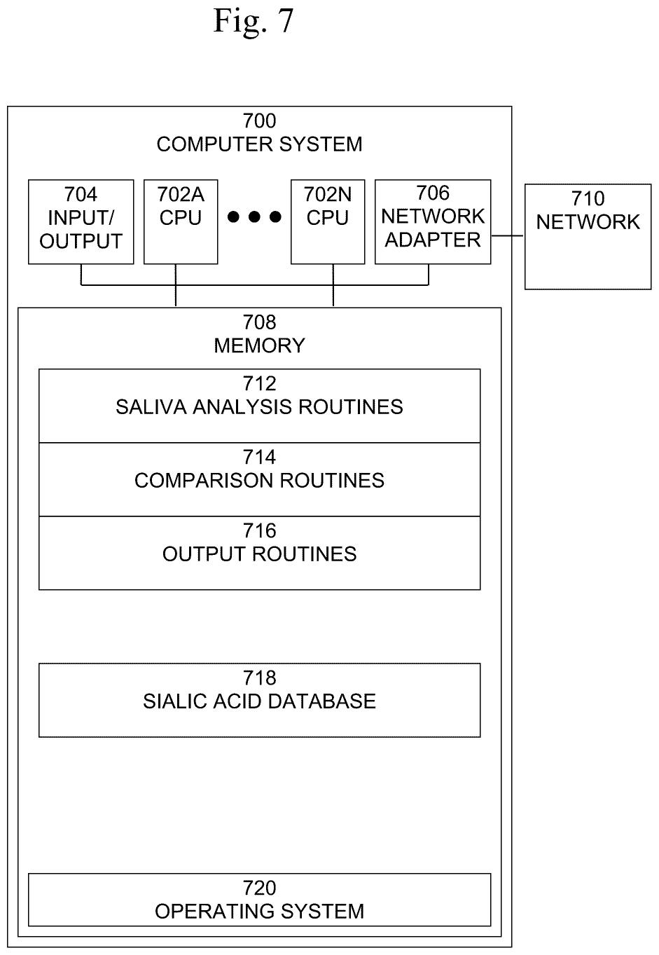

An exemplary block diagram of a computing device 700, in which processes involved in the embodiments described herein may be implemented, is shown in FIG. 7. Computing device 700 may be a programmed general-purpose computer system, such as an embedded processor, microcontroller, system on a chip, microprocessor, smartphone, tablet, or other mobile computing device, personal computer, workstation, server system, and minicomputer or mainframe computer. Computing device 700 may include one or more processors (CPUs) 702A-702N, input/output circuitry 704, network adapter 706, and memory 708. CPUs 702A-702N execute program instructions in order to carry out the functions of the present invention. Typically, CPUs 702A-702N are one or more microprocessors, such as an INTEL PENTIUM® processor, etc. FIG. 7 illustrates an embodiment in which computing device 700 is implemented as a single multi-processor computer system, in which multiple processors 702A-702N share system resources, such as memory 708, input/output circuitry 704, and network adapter 706. However, the present invention also contemplates embodiments in which computing device 700 is implemented as a plurality of networked computer systems, which may be single-processor computer systems, multi-processor computer systems, or a mix thereof.

Input/output circuitry 704 provides the capability to input data to, or output data from, computing device 700. For example, input/output circuitry may include input devices, such as keyboards, mice, touchpads, trackballs, scanners, etc., output devices, such as video adapters, monitors, printers, etc., and input/output devices, such as, modems, etc. Network adapter 706 interfaces device 700 with a network 710. Network 710 may be any public or proprietary LAN or WAN, including, but not limited to the Internet.

Memory 708 stores program instructions that are executed by, and data that are used and processed by, CPU 702 to perform the functions of computing device 700. Memory 708 may include, for example, electronic memory devices, such as random-access memory (RAM), read-only memory (ROM), programmable read-only memory (PROM), electrically erasable programmable read-only memory (EEPROM), flash memory, etc., and electro-mechanical memory, such as magnetic disk drives, tape drives, optical disk drives, etc., which may use an integrated drive electronics (IDE) interface, or a variation or enhancement thereof, such as enhanced IDE (EIDE) or ultra-direct memory access (UDMA), or a small computer system interface (SCSI) based interface, or a variation or enhancement thereof, such as fast-SCSI, wide-SCSI, fast and wide-SCSI, etc., or Serial Advanced Technology Attachment (SATA), or a variation or enhancement thereof, or a fiber channel-arbitrated loop (FC-AL) interface.

The contents of memory 708 may vary depending upon the function that computing device 700 is programmed to perform. One of skill in the art would recognize that these routines, along with the memory contents related to those routines, may typically be included on one system or device, but or may be distributed among a plurality of systems or devices, based on well-known engineering considerations. The present invention contemplates any and all such arrangements.

In the example shown in FIG. 7, memory 708 may include saliva analysis routines 712, comparison routines 714, output routines 716, sialic acid database 718, and operating system 720. For example, saliva analysis routines 712 may include routines that interact with one or more analysis devices to determine the sialic acid concentration in a saliva sample, using one of the techniques described above, or other suitable technique. Comparison routines 714 may include routines to compare the determined sialic acid concentration with a database relating sialic acid concentrations with cancer conditions. Output routines 716 may include routines to generate and output a probability of presence of particular cancer conditions. Sialic acid database 718 may include data relating sialic acid concentrations with cancer conditions. Operating system 720 provides overall system functionality.

As shown in FIG. 7, the present invention contemplates implementation on a system or systems that provide multi-processor, multi-tasking, multi-process, and/or multi-thread computing, as well as implementation on systems that provide only single processor, single thread computing. Multi-processor computing involves performing computing using more than one processor. Multi-tasking computing involves performing computing using more than one operating system task. A task is an operating system concept that refers to the combination of a program being executed and bookkeeping information used by the operating system. Whenever a program is executed, the operating system creates a new task for it. The task is like an envelope for the program in that it identifies the program with a task number and attaches other bookkeeping information to it. Many operating systems, including Linux, UNIX®, OS/2®, and Windows®, are capable of running many tasks at the same time and are called multitasking operating systems. Multi-tasking is the ability of an operating system to execute more than one executable at the same time. Each executable is running in its own address space, meaning that the executables have no way to share any of their memory. This has advantages, because it is impossible for any program to damage the execution of any of the other programs running on the system. However, the programs have no way to exchange any information except through the operating system (or by reading files stored on the file system). Multi-process computing is similar to multi-tasking computing, as the terms task and process are often used interchangeably, although some operating systems make a distinction between the two.

The present invention may be a system, a method, and/or a computer program product at any possible technical detail level of integration. The computer program product may include a computer readable storage medium (or media) having computer readable program instructions thereon for causing a processor to carry out aspects of the present invention. The computer readable storage medium can be a tangible device that can retain and store instructions for use by an instruction execution device.

The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry (such as that shown at 208 of FIG. 2) may include, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Although specific embodiments of the present invention have been described, it will be understood by those of skill in the art that there are other embodiments that are equivalent to the described embodiments. Accordingly, it is to be understood that the invention is not to be limited by the specific illustrated embodiments, but only by the scope of the appended claims. Further, it is to be noted that, as used in the claims, the term coupled may refer to electrical or optical connection and may include both direct connection between two or more devices and indirect connection of two or more devices through one or more intermediate devices.

What is claimed is:

1. A computer-implemented method for determining a probability of presence of a cancer condition comprising:
determining a concentration of sialic acid in a saliva sample of a person using a sialic acid concentration analysis device comprising:
receiving the saliva sample of the person and processing the saliva sample of the person for analysis, analyzing the saliva sample of the person to determine the sialic acid concentration, and
generating digital data representing the concentration of sialic acid in the saliva sample of the person;
receiving the digital data representing the concentration of sialic acid in the saliva sample of the person;
comparing the determined concentration of sialic acid with a database relating sialic acid concentrations with cancer conditions; and
determining and outputting a probability of presence of a particular cancer condition based on the comparison of the determined concentration of sialic acid with a database relating sialic acid concentrations with cancer conditions.

2. The method of claim 1, wherein the sialic acid concentration analysis device comprises a spectrophotometer.

3. The method of claim 1, wherein the sialic acid concentration analysis device comprises a Raman spectrometer.

4. The method of claim 1, wherein the sialic acid concentration analysis device comprises an electrochemical sensor.

5. A system for determining a probability of presence of a cancer condition comprising:
a sialic acid concentration analysis device adapted to determine a concentration of sialic acid in a saliva sample of a person comprising:
apparatus configured to receive the saliva sample of the person and process the saliva sample of the person for analysis,
apparatus configured to analyze the saliva sample of the person to determine the sialic acid concentration, and
circuitry configured to generate digital data representing the concentration of sialic acid in the saliva sample of the person;
a computer system comprising a processor, memory accessible by the processor, and program instruction and data stored in the memory whereby the computer system is adapted to:
control the sialic acid concentration analysis device so as to obtain the concentration of sialic acid, receive the digital data representing the concentration of sialic acid in the saliva sample of the person,
compare the determined concentration of sialic acid with a database relating sialic acid concentrations with cancer conditions, and
determine and output a probability of presence of a particular cancer condition based on the comparison of the determined concentration of sialic acid with a database relating sialic acid concentrations with cancer conditions.

6. The method of claim 5. wherein the sialic acid concentration analysis device comprises a spectrophotometer.

7. The method of claim 5, wherein the sialic acid concentration analysis device comprises a Raman spectrometer.

8. The method of claim 5, wherein the sialic acid concentration analysis device comprises an electrochemical sensor.
